(19) United States
(12) Patent Application Publication
(10) Pub. No.: US 2008/0255578 A1
(43) Pub. Date: Oct. 16, 2008

(54) CORNEAL ENDOTHELIAL TISSUE INSERTER

(75) Inventor: WILLIAM NEUSIDL, Phillipsburg, NJ (US)

Correspondence Address: MICHAEL RIES, 318 PARKER PLACE, OSWEGO, IL, 60543, US

(73) Assignee: FISHER SURGICAL, Arnold, MO (US)

(21) Appl. No.: 12/027,822

(22) Filed: Feb. 7, 2008

Related U.S. Application Data
(60) Provisional application No. 60/889,236, filed on Feb. 9, 2007.

(51) Int. Cl. A61F 9/007

(52) U.S. Cl. 606/107

(57) ABSTRACT

The present invention is a an instrument to be utilized in a procedure to transfer donor endothelial cells to the cornea without injuring or destroying cells during the transfer process. Currently there are no instruments/techniques that facilitate the transfer process. Techniques today include forcing the tissue through a 3-5 mm opening with forceps that crush and damage the tissue. My instrument would allow for the transfer of living endothelial cells with minimal or no trauma.

This application claims priority to U.S. Provisional Application 60/889,236 filed Feb. 09, 2007, the entire disclosure of which is incorporated by reference.

### TECHNICAL FIELD & BACKGROUND

The present invention generally relates to a medical device to work with eye tissue. More specifically, the present invention is a device used to retract an extremely thin layer of donor endothelial eye tissue and inject this tissue within a recipient's eye.

The present invention is a corneal endothelial tissue inserter. This transfer apparatus consists of a reusable, hollow rigid tube/barrel that has a disposable tissue holder/transfer chamber at its distal end. This soft, plastic platform-paddle is connected to the barrel by a thin rod/tube that can be advanced forward or retracted back into the barrel by turning a knob at the proximal end. This knob works by a threaded system and allows the paddle to be advanced or retracted at multiple positions while holding the instrument. The platform could also be advanced or retracted by application of sliding the flow regulator ring forward or backwards. The flow regulator ring is connected to a rod with a lumen. This in turn is connected to the disposable platform which will house and protect the tissue during the transfer process.

Turning the knob to retract the paddle into the barrel will result in the soft plastic paddle conforming to the walls of the barrel, folding the paddle into a cylinder slightly smaller in diameter as the internal diameter of the distal end of the tapered barrel opening. This will fold the corneal endothelial tissue seated on the inside of the paddle in a gentle manner with no acute folds. Depending on the diameter of the paddle, one could fold the tissue using the bifold (60/40) or trifold (“taco”) techniques. Since the tissue is transferred with out forceps and there is no forcing the tissue through a small incision, these folding techniques are relatively safe to use. The paddle will fold over the tissue and protect the vital cells during the transfer process through a small incision. The paddle is made of a flexible soft plastic and can be made in different diameters to facilitate transplantation through different size incisions. Folding the tissue may be necessary to produce a transfer device that enables the use of a smaller paddle and allow for smaller incisions. The soft sheet of flexible plastic will surround the tissue and therefore offer protection during the transfer process, i.e. no crushing or squeezing through the incision as in forceps transfer. After retracting the paddle that now houses and protects the tissue, the distal, tapered end of the barrel is inserted into the incision in the eye. Rotating the knob to extend the rod will result in the unfolding of the plastic paddle thereby allowing the tissue to release and naturally unfold. The irrigation, controlled with the flow regulator, could be used in the event that the tissue sticks to the paddle or resists unfolding once the paddle is open or to maintain the anterior chamber during transfer.

The above explanation explains how the instrument is used, as well as the procedure involved, to transfer the endothelial cells through a small corneal incision. Different size platforms to house the tissue may be used during the tissue transfer process. Irrigation may be added to the instrument with a flow regulator. Different ways of retracting the platform within the protective barrel are possible including threads or push rod.

### BRIEF DESCRIPTION OF THE DRAWINGS

The present invention will be described by way of exemplary embodiments, but not limitations, illustrated in the accompanying drawings in which like references denote similar elements, and in which:

FIG. 1 illustrates a drawing of an instrument, in accordance with one embodiment of the present invention;

FIG. 2 illustrates a drawing of an instrument, in accordance with one embodiment of the present invention;

FIG. 3 illustrates a drawing of an instrument, in accordance with one embodiment of the present invention;

FIG. 4 illustrates a drawing of an instrument, in accordance with one embodiment of the present invention;

FIG. 5a illustrates a drawing of an instrument, in accordance with one embodiment of the present invention;

FIG. 5b illustrates a drawing of an instrument, in accordance with one embodiment of the present invention

FIG. 6a illustrates a drawing of an instrument, in accordance with one embodiment of the present invention

FIG. 6b illustrates a drawing of an instrument, in accordance with one embodiment of the present invention

FIG. 6c illustrates a drawing of an instrument, in accordance with one embodiment of the present invention; and

FIG. 7 illustrates a drawing of an instrument, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

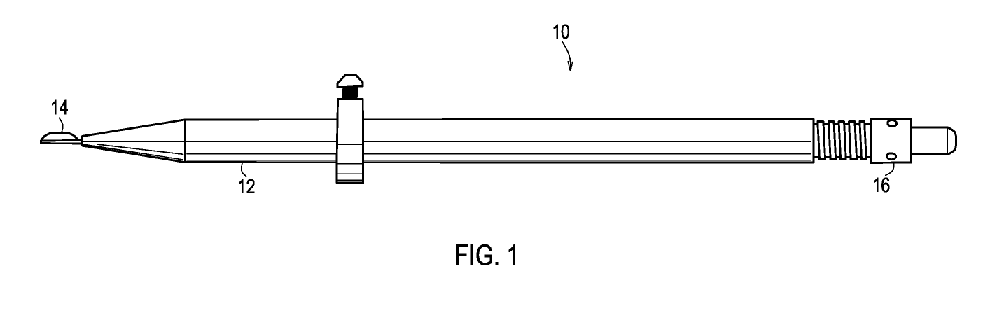

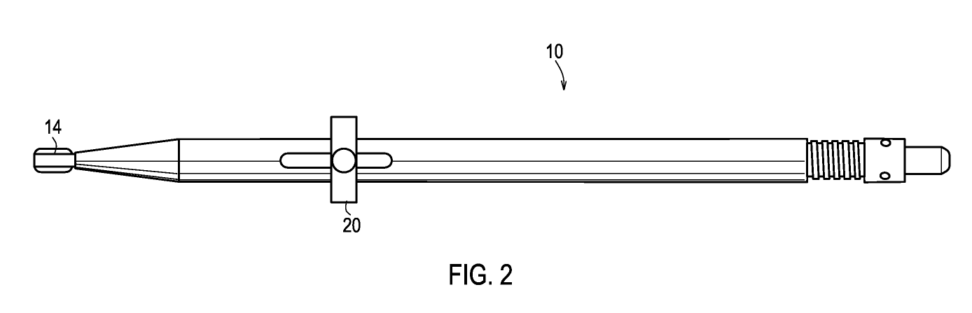

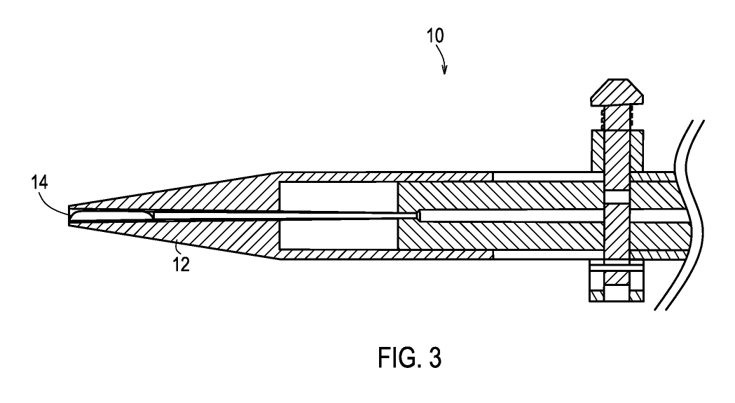

Referring now to FIG. 1, as in one embodiment illustrated is a side view of an instrument 10. Shown is a hollow rigid tube or barrel 12 with a disposable tissue holder or transfer chamber 14 at a distal end of the instrument. The holder 14 may be advanced forward or retracted back into the barrel 12 by turning a knob 16 at a proximal end of the barrel 12. Referring to FIG. 2 as in one embodiment shown is a top view of the instrument 10. A flow regulator ring 20 is shown that can be moved forward or backwards to advance or retract the tissue holder or platform 14. Referring to FIG. 3 shown is a side sectional view of instrument 1 0 with the holder 14 retracted all the way into barrel 12.

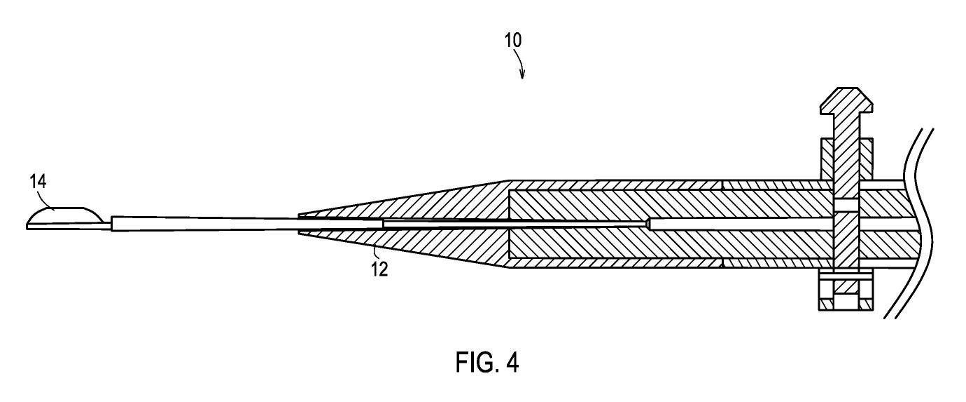

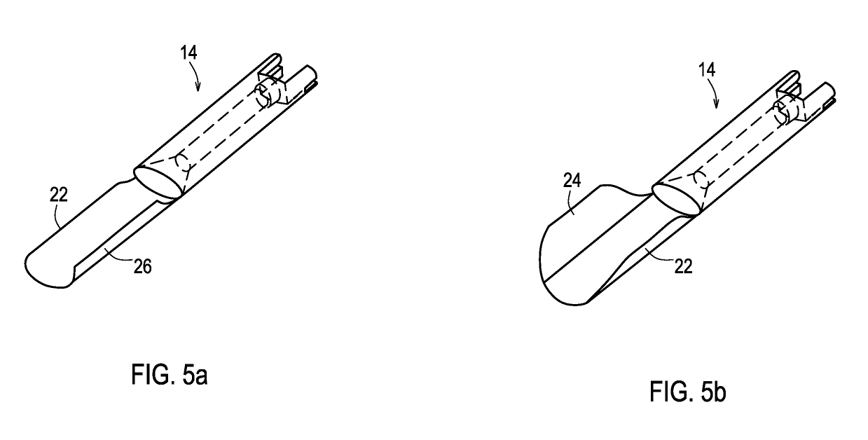

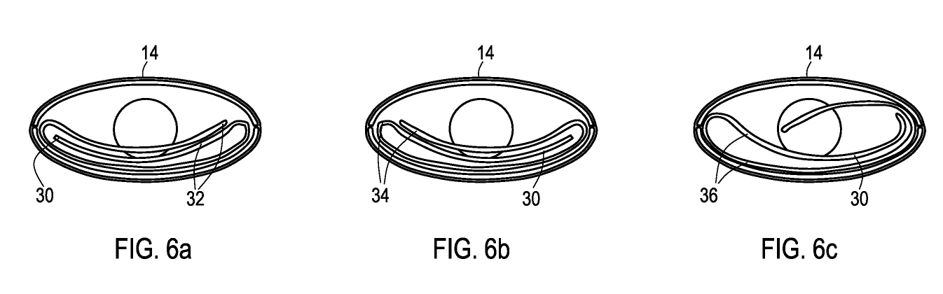

Referring to FIG. 4 as in one embodiment a side view of instrument 10 is shown with the holder 14 advanced all the way forward of the barrel 12. Referring to FIGS. 5a and 5b as in both embodiments shown is tissue holder 14 as the tissue holder 14 is retracted and advanced the tissue holder flaps 22 will be in a paddle open position 24 and paddle closed position 26. The tissue holder flaps 22 will conform to the walls of the barrel 12 and form a cylinder shape to easily go inside barrel 12. Referring to FIGS. 6a, 6b and 6c as in all three embodiments shown is an end view of tissue holder 14, in FIG. 6a shown is folding a corneal endothelial tissue 30 into a trifold arrangement 32. In FIG. 6b shown is folding a corneal endothelial tissue 30 into a reverse trifold arrangement 34. In FIG. 6c shown is folding a corneal endothelial tissue 30 into a bifold arrangement 36.

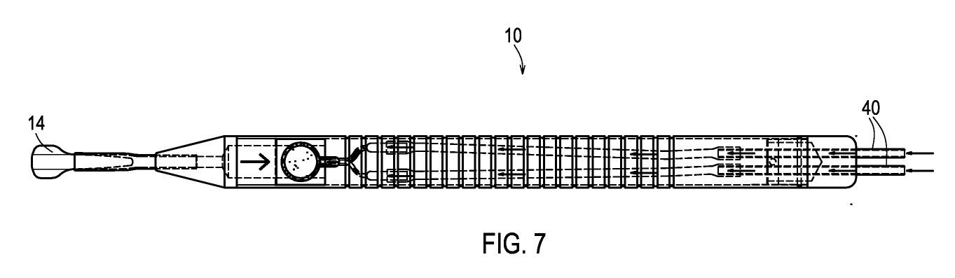

Referring to FIG. 7 as in one embodiment a top view of instrument 10 is shown with irrigation channels 40 to provide irrigation to the paddle 14 in the event that the tissue sticks to the paddle 14 or the tissue resists unfolding once the paddle 14 is open or can be used to maintain the anterior chamber during the transfer of tissue into the eye.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

What is claimed is:

1. An instrument comprising:
a disposable platform that tissue would rest on, the platform would be connected to a barrel into which the tissue would be retracted a barrel end would be tapered to allow the transfer of endothelial cells through a small incision with substantially little trauma, the platform would be made of a thin plastic polymer that would fold around the tissue in a cylindrical shape once retracted into the barrel.

2. The instrument of claim 1 wherein turning a knob to retract the platform into the barrel will result in the platform conforming to the walls of the barrel, folding the platform into a cylinder slightly smaller in diameter as an internal diameter of a distal end of a tapered barrel opening, folding the tissue seated on an inside of the paddle in a gentle manner with no acute folds, depending on the diameter of the platform, one would fold the tissue using a selected one of a bifold and no folds technique.

3. The instrument of claim 1 wherein turning a knob to retract the platform into the barrel will result in the platform conforming to the walls of the barrel, folding the platform into a cylinder slightly smaller in diameter as an internal diameter of a distal end of a tapered barrel opening, folding the tissue seated on an inside of the paddle in a gentle manner with no acute folds, depending on the diameter of the platform, one would fold the tissue using a trifold technique.

4. The instrument of claim 1 wherein the platform is advanced or retracted by sliding a flow regulator ring forward or backwards.

5. The instrument of claim 4 wherein, the flow regulator ring is connected to a rod with a lumen which extends to the platform and may be utilized to maintain an anterior chamber during transfer, this in turn is connected to the platform which will house and protect the tissue during the transfer process.

6. An instrument comprising:
a one piece design in which the entire device would be utilized as a disposable injector after each use a platform that tissue would rest on, the platform would be connected to an irrigating lumen into which the tissue would be retracted into a barrel, a barrel end would be tapered to allow the transfer of endothelial cell tissue through a small incision with substantially little or no trauma, the platform would be made of a thin plastic polymer that would fold around the tissue in a cylindrical shape once retracted into the barrel.

7. The instrument of claim 6 wherein turning a knob to retract the platform into the barrel will result in the platform conforming to the walls of the barrel, folding the platform into a cylinder slightly smaller in diameter as an internal diameter of a distal end of a tapered barrel opening, folding the tissue seated on an inside of the paddle in a gentle manner with no acute folds, depending on the diameter of the platform, one would fold the tissue using a selected one of a bifold technique and no folds.

8. The instrument of claim 6 wherein turning a knob to retract the platform into the barrel will result in the platform conforming to the walls of the barrel, folding the platform into a cylinder slightly smaller in diameter as an internal diameter of a distal end of a tapered barrel opening, folding the tissue seated on an inside of the paddle in a gentle manner with no acute folds, depending on the diameter of the platform, one would fold the tissue using a trifold technique.

9. The instrument of claim 6 wherein the platform is advanced or retracted by sliding a flow regulator ring or button forward or backwards.

10. The instrument of claim 9 wherein, the flow regulator ring or button is connected to a rod with a lumen, this in turn is connected to the platform which will house and protect the tissue during the transfer process.
